(19) United States
(12) Patent Application Publication
(10) Pub. No.: US 2003/0069550 A1
(43) Pub. Date: Apr. 10, 2003

(54) Hypodermic needle

(76) Inventor: M. Keith Sharp, Jeffersonville, IN (US)

Correspondence Address: JANET A. PIOLI, BRINKS HOFER GILSON & LIONE, 455 NORTH CITYFRONT PLAZA DRIVE, CHICAGO, IL, 60611, US

(21) Appl. No.: 09/973,477

(22) Filed: Oct. 6, 2001

(51) Int. Cl. A61M 5/32

(52) U.S. Cl. 604/272; 600/576

(57) ABSTRACT

A hypodermic needle includes a hollow tube having an angled end with respect to a longitudinal axis of the tube, the end having an opening surrounded by an external peripheral rim. At least a portion of the external peripheral rim is beveled back at least 25%, to form an internal beveled surface.

### BACKGROUND

The present invention relates generally to hypodermic needles, and more particularly to hypodermic needles for collecting blood samples for laboratory testing that minimize hemolysis of red blood cells in the sample.

The quality of the information derived from a laboratory blood test depends greatly on the proper collection of the specimen submitted for analysis. Many conventional devices are available for obtaining blood samples from a patient for laboratory testing. Typically, a syringe or a vacuum tube, and a conventional hypodermic needle, are used to draw blood from a patient. Conventional hypodermic needles 10, such as those shown in FIGS. 1-3, typically include a hollow shaft having a cylindrical inner surface 12, a cylindrical outer surface 14 and, a piercing part 16 that is a more or less sharply angled wedge at one end of the needle. The piercing part 16, as shown in top view in FIGS. 2 and 3, appears as an oval opening 18 in line with the bore 20 of the needle 10. An outer peripheral rim 22 surrounds the opening 18. The outer peripheral rim 22 has a generally planar surface with the outer edge 24 and inner edge 26 of the rim 22 forming cutting edges. Such hypodermic needles are commonly used to withdraw blood from individuals for diagnostic purposes.

A problem often encountered with conventional hypodermic needles is hemolysis. Hemolysis occurs when the membrane surrounding red blood cells is ruptured and hemoglobin and other intracellular components escape into the serum or plasma. Serum or plasma with various concentrations of plasma-free hemoglobin varies in color from faint pink to bright red, whereas normal serum or plasma has a straw color. Hemolyzed specimens exhibit anomalously increased levels of potassium, LDH, AST, ALT, phosphorous, magnesium and ammonia. Even slight hemolysis may alter certain test results, including those for glucose, creatinine, bilirubin and alkaline phosphatase, leading to inaccurate test results and the need for repeated blood draws. The incidence of hemolysis is particularly great in clinics taking a large number of samples, and particularly in emergency departments, where timely and accurate blood test results may be critical for diagnosis.

Hemolysis may occur with a syringe and hypodermic needle, if the syringe plunger is drawn back with too much force, thereby creating fluid stresses in the blood as it is drawn into the needle. Similarly, with vacuum tube devices, the fixed vacuum of the specimen tube may draw blood too quickly through the needle. It has been thought that the force of the vacuum or drawback of the plunger creates fluid stresses, which may cause red blood cells to shear and break.

It has been suggested that hemolysis can be minimized in conventional syringes and needles by slowing the drawback of the syringe plunger or by providing vacuum tubes with reduced vacuum. Both methods are time-consuming and, in the case of vacuum tubes with reduced vacuum, additional or larger vacuum tubes are needed to withdraw the desired amount of blood, increasing the expense of blood collection.

Other attempts have been made to modify conventional blood collection devices to minimize hemolysis. For example, U.S. Pat. No. 5,133,362, issued to Moss, discloses a blood collection needle that uses a smaller gauge needle for introduction into the patient than is used for the specimen tubes. The device uses smaller gauge needles to provide improved peripheral access, while also slowing blood flow through the needles to reduce hemolysis. Use of this device also slows the blood collection process and, further, may present problems in obtaining samples from certain patients, such as young or elderly individuals, whose veins are smaller and more susceptible to collapse.

### SUMMARY

The scope of the invention is determined solely by the appended claims and their equivalents and is not affected to any degree by the statements within this summary. The invention provides a hypodermic needle for the collection of a fluid sample, preferably a blood sample, either percutaneously or from an indwelling catheter. Specifically, the hypodermic needle of the present invention addresses the disadvantages of conventional needles by providing a hypodermic needle having an entrance that facilitates uniform fluid flow and reduces fluid stresses at the entrance of the needle and minimizes hemolysis during the blood collection.

In accordance with one aspect of the present invention, a hypodermic needle includes an internal substantially cylindrical surface, an external substantially cylindrical surface, and an end formed at an angle with respect to the longitudinal axis of the needle. The needle has an oval opening in line with the bore of the needle and sharpened point at the end of the oval. An exterior planar rim having an external edge and an internal edge partially or wholly extends around the circumference of the opening. At least a portion of the rim is beveled inwardly toward the interior of the needle to form a beveled surface that faces the interior of the needle and extends partially around the inner circumference of the entrance of the needle opening.

In one embodiment, the exterior rim extends around the full circumference of the opening but decreases in width with the degree of internal beveling.

In a preferred embodiment, the entire surface of the rim in one region, preferably the region opposed to the piercing point, has been beveled back to form a bell-mouthed or frustoconical entrance to the needle in that region.

### BRIEF DESCRIPTION OF SEVERAL VIEWS OF THE DRAWINGS

FIG. 1 is a top view of a conventional hypodermic needle.

FIG. 2 is a frontal perspective view of a conventional hypodermic needle.

FIG. 3 is a cross-sectional view of a conventional hypodermic needle.

FIG. 4 is a top view an embodiment of a hypodermic needle according to the present invention.

FIG. 5 is a frontal perspective view of an embodiment of a hypodermic needle according to the present invention.

FIG. 6 is a cross-sectional view of an embodiment of a hypodermic needle according to the present invention.

FIG. 7 is a top view of an embodiment of a hypodermic needle according to the present invention.

FIG. 8 is a cross-sectional view an embodiment of a hypodermic needle according to the present invention.

FIG. 9 is a cross-sectional view of the hypodermic needle of FIG. 8 inserted into a vein.

FIG. 10 is a top view an embodiment of a hypodermic needle according to the present invention.

FIG. 11 is a cross-sectional view of an embodiment of a hypodermic needle according to the present invention.

FIG. 12 is a cross-sectional view of an embodiment of a hypodermic needle according to the present invention.

FIG. 13 is a cross-sectional view of an embodiment of a hypodermic needle according to the present invention.

FIG. 14 is a top view of an embodiment of a hypodermic needle according to the present invention.

FIG. 15 is a cross-sectional view of iso-velocity contours of fluid in a conventional hypodermic needle.

FIG. 16 is a cross-sectional view of iso-velocity contours of fluid in an embodiment of a hypodermic needle according to the present invention.

FIG. 17 is a cross-sectional view of iso-velocity contours of fluid in another embodiment of a hypodermic needle according to the present invention.

FIG. 18 is a cross-sectional view of an embodiment of a hypodermic needle according to the present invention.

FIG. 19 is a top view of an embodiment of a hypodermic needle according to the present invention.

FIG. 20 is a top view of an embodiment of a hypodermic needle according to the present invention.

### DETAILED DESCRIPTION

We have discovered that with conventional needles there is a problem of strongly non-uniform fluid flow at the entrance of the needle. With non-uniform flow, significantly increased normal and shear stresses result at the entrance of the needle. Previous methods or devices have not addressed the issue of fluid stress concentration at the entrance of the needle. The present invention makes use of the discovery that fluid stresses at the entrance of a hypodermic needle may be reduced and fluid flow made more uniform by properly altering the shape of the entrance to the needle.

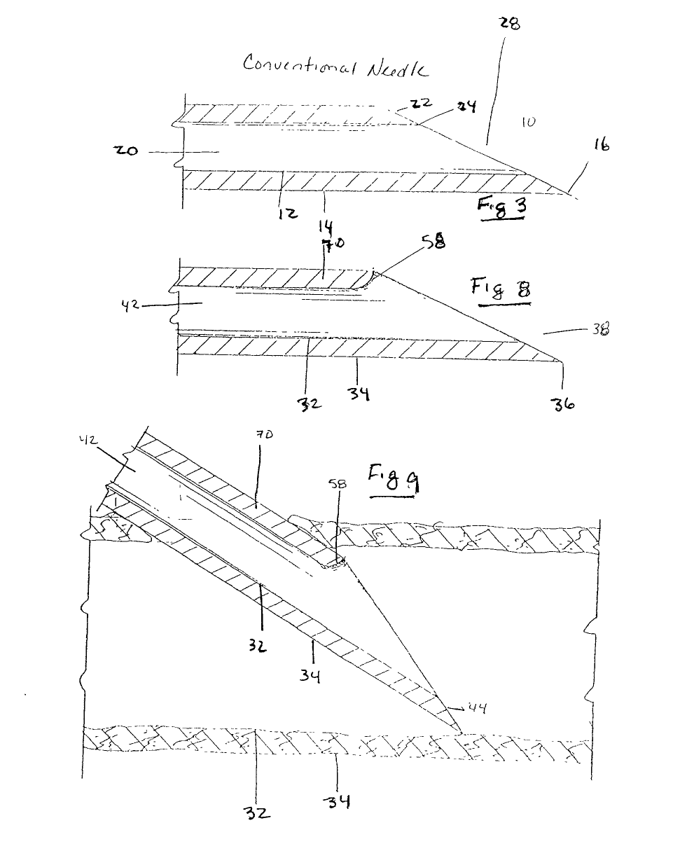

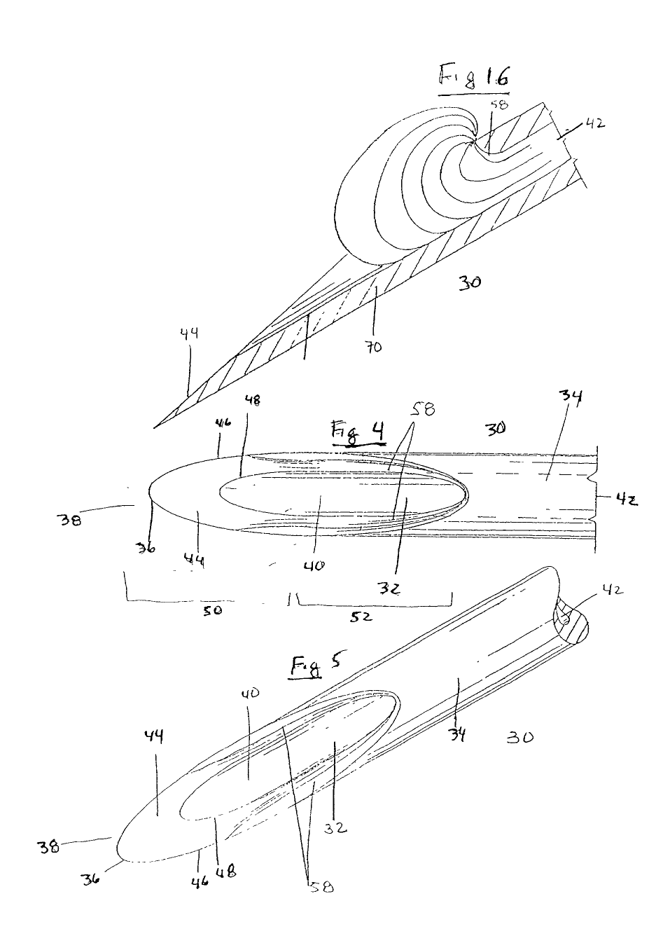

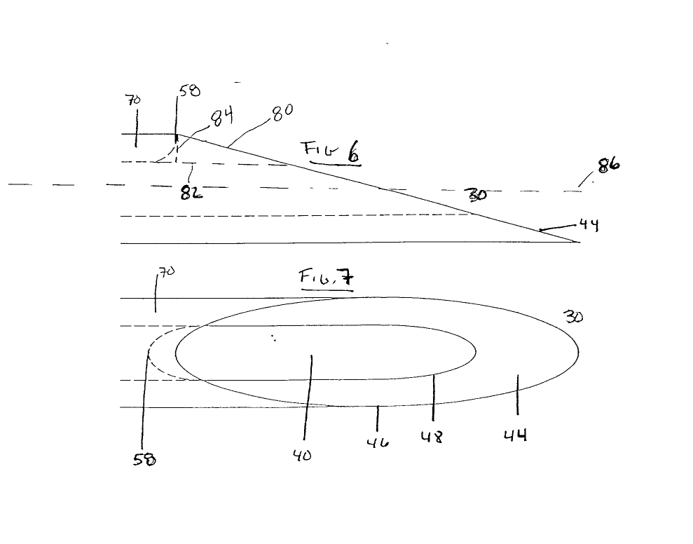

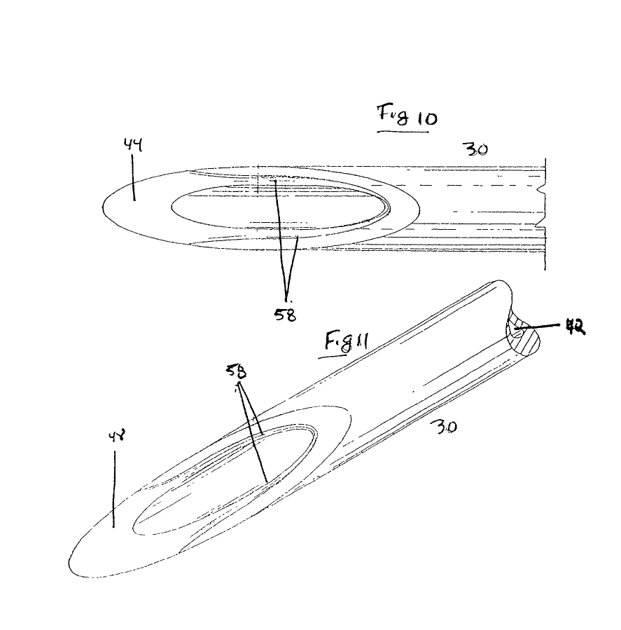

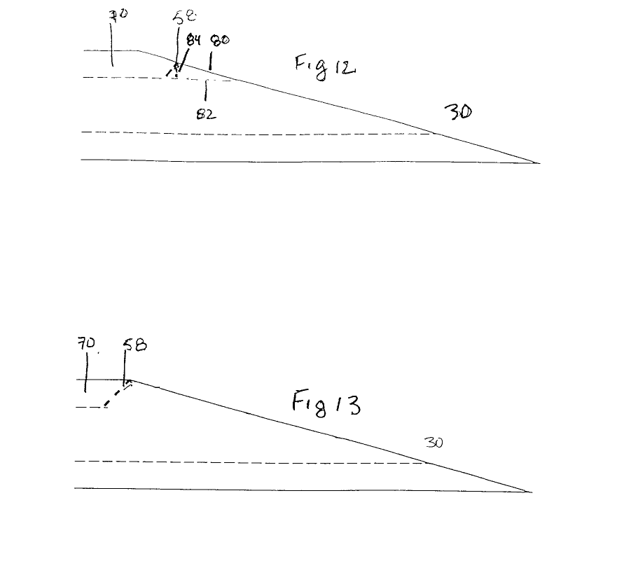

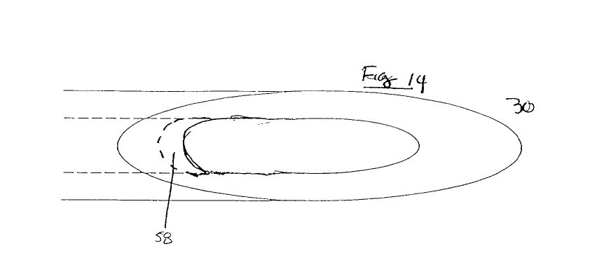

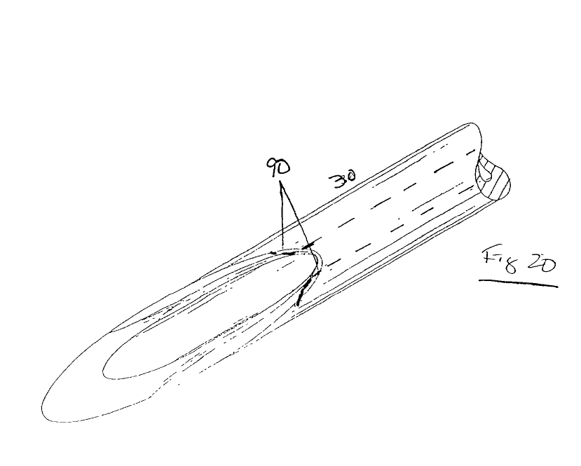

FIGS. 4-14 and 18-20 show a hypodermic needle 30 according to one embodiment of the present invention. As illustrated, the hypodermic needle 30 is comprised of a cylindrical hollow shaft or tube having a substantially cylindrical interior surface 32, a substantially cylindrical outer surface 34 and an angled piercing or cutting point 36 at the end 38 of the needle 30. As shown in FIG. 6, the end 38 of the needle 30 is formed at an angle with respect to the longitudinal axis 86 of the needle 30. The end 38 of the needle 30 may be formed by first cutting a cylindrical shaft at an angle of, preferably about 10 to about 50 degrees, preferably from about 12 degrees to about 25 degrees, and more preferably from about 13 degrees to about 18 degrees with respect to the longitudinal axis of the needle 30 to form a needle having a planar angle cut end 38. The end 38 of the needle shown in top view in FIGS. 4 and 5 has an oval opening 40 that is in line with the bore 42 of the needle 30.

As shown in FIG. 5, generally planar external peripheral rim 44 having an outer edge 46 and an inner edge 48, front half and rear half regions 50 and 52, respectively, surrounds the opening 40 and connects the internal and external cylindrical surfaces 32 and 34 of the needle 30.

In conventional needles, shown in FIGS. 2 and 3, the external peripheral rim 22 surrounds the entire circumference of the opening 18. Due to the angling of the end 28, the inner edge 24 of the rim 22 presents a cutting edge around the entrance of the needle 10. As is most clearly shown in FIG. 3, the inner cutting edge 24 is particularly pronounced at the entrance in the rear region 27 of the end 28 of the needle 10.

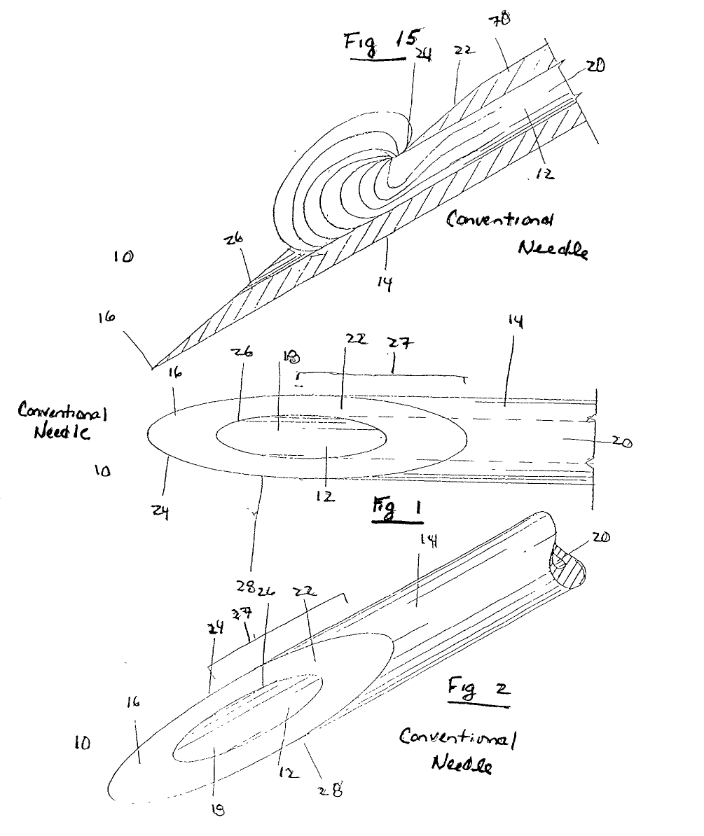

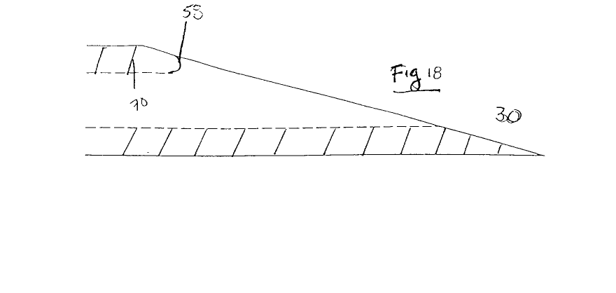

Our studies and modeling have shown that in conventional needles such as those shown in FIGS. 1, 2, and 3, the inner edge 24 of the rim 22 causes or contributes to an increase of non-uniform fluid flow and significant gradients in normal and shear stresses at the entrance to the needle opening. FIG. 15 partially illustrates the non-uniform flow of fluid at the entrance of the opening of a conventional needle around internal edge 24.

In the present invention, as shown in FIGS. 4 and 5, the rim 44 and the inner edge 48 which form a cutting edge in conventional needles, is beveled back to form an internal beveled surface 58 that at least partially surrounds the inner periphery of the opening 40. The internal beveled surface 58 is usually located in the rear half region 52, and may extend into the front half region. The internal beveled surface 58 surrounds preferably five percent to eighty-five percent of the inner periphery of the opening 40, more preferably from twenty percent to seventy percent of the inner periphery of the opening 40, and most preferably from thirty percent to seventy percent of the inner periphery of the opening 40.

The end 38 of the needle 30, as set forth above, is formed by first cutting a cylindrical shaft preferably at an angle of from 10 degrees to 45 degrees, more preferably from 12 degrees to 25 degrees, and most preferably from 13 degrees to 18 degrees, with respect to the longitudinal axis (not shown) of the needle 30 to form the angled end 38. Thereafter, the rim 44 may be partially or fully beveled toward the interior surface 32 of the needle 30 by, for example, a milling cutter, or similar device, to form the internal beveled surface 58. The hypodermic needle in accordance with the present invention can be formed from conventional materials, such as steel, medical grade plastics, metal alloys, composites, ceramics, or like materials.

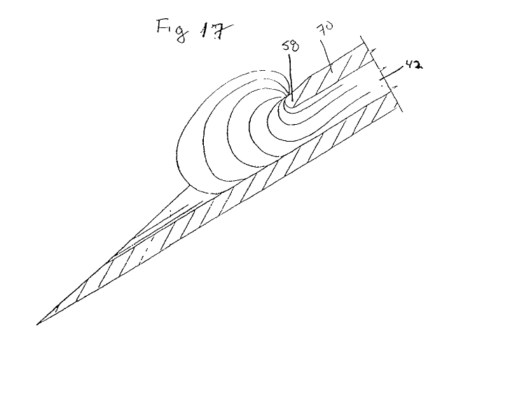

The internal beveled surface 58 may present a straight surface 60 as shown in FIG. 12 and 13, or a convex surface curved by varying degress toward the interior of the needle, as shown in FIGS. 6, 8, 9, 16 and 18. The degree of beveling back is preferably at least 25% of the thickness of the wall 70 of the needle 30, and may be 30, 35, 40, 45, 50, 55, 60, 65, 70, 75, 80, 85, 90, 95 or 100%. The degree of beveling back is the distance 84, as a percentage of the wall thickness, between the original external surface 80 and the original internal surface 82 at the farthest point of the beveled surface 58, as shown in FIGS. 6 and 12.

When the internal beveled surface 58 is curved, as may be obtained with a round-over milling cutter, a circle coincident with the curvature of the beveled portion has a radius of curvature that is preferably at least 25% of the thickness of the wall of the needle, and may be 30, 35, 40, 45, 50, 55, 60, 65, 70, 75, 80, 85, 90, 95 or 100% of the thickness of the wall of the needle. Preferably, a circle coincident with the curvature of the beveled portion has a radius of curvature that is from 30 to 100%, more preferably from 50% to 100%, and most preferably from 75% to 100% of the thickness of the wall of the needle.

When the internal beveled surface 58 is curved, the entrance of the needle has a partial bell-mouth configuration. In this configuration, as well as when the internal beveled surface is not curved, the interior space of the needle in the region of beveling is frustoconical as shown by the dotted lines 90 in FIG. 20.

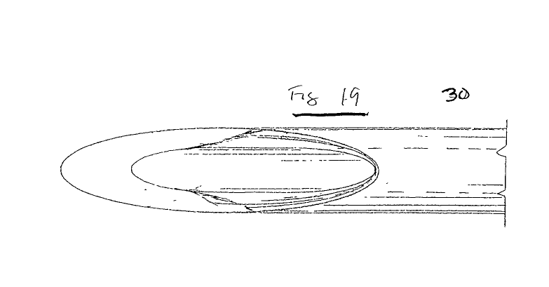

Modeling has shown, as partially illustrated in FIGS. 15, 16, 17, that as the degree of beveling increases, the fluid flow at the entrance of the needle becomes more uniform and the fluid stresses decrease. Our studies indicate that the increased uniformity of fluid flow at the entrance and the decrease in fluid stresses should contribute significantly to reduced stress on the membrane of red blood cells and, consequently, a reduction in hemolysis.

Of course, it should be understood that a wide range of changes and modifications can be made to the embodiments described above. It is therefore intended that the foregoing description illustrates rather than limits this invention, and that it is the following claims, including all equivalents, that define this invention.

1. A hypodermic needle, comprising:
a hollow tube having an angled end with respect to a longitudinal axis of the tube, the end having an opening surrounded by an external peripheral rim,
wherein at least a portion of the external peripheral rim is beveled back at least 25%, to form an internal beveled surface.

2. The hypodermic needle of claim 1, wherein the at least a portion of the external peripheral rim is beveled back at least 30%.

3. The hypodermic needle of claim 1, wherein the at least a portion of the external peripheral rim is beveled back at least 35%.

4. The hypodermic needle of claim 1, wherein the at least a portion of the external peripheral rim is beveled back at least 40%.

5. The hypodermic needle of claim 1, wherein the at least a portion of the external peripheral rim is beveled back at least 50%.

6. The hypodermic needle of claim 1, wherein the internal beveled surface is curved.

7. The hypodermic needle of claim 1, wherein a circle coincident with the curvature of the internal beveled surface has a radius of curvature that is at least 25% of a thickness of a wall of the needle.

8. The hypodermic needle of claim 1, wherein the internal beveled surface surrounds 5-85% of the opening.

9. The hypodermic needle of claim 1, wherein the internal beveled surface surrounds 20-70% of the opening.

10. A hypodermic needle, comprising:
a hollow tube having an angled end with respects to a longitudinal axis of the tube, the end having an opening surrounded by an external peripheral rim, and
means for reducing fluid stress at the entrance of the needle.

11. In a hypodermic needle having an internal substantially cylindrical surface; an external substantially cylindrical surface; an end angled with respect to a longitudinal axis of the needle, the end having an opening and defining a piercing tip; an outer peripheral rim, the rim partially surrounding a first region of the opening proximal to the piercing tip and connecting the external and internal cylindrical surfaces of the needle; the improvement comprising an internal beveled surface on the internal surface of the needle partially surrounding a second region of said opening opposite the first region, wherein the degree of beveling back of the rim is at least 25%.

12. The hypodermic needle of claim 11, wherein the at least a portion of the external peripheral rim is beveled back at least 30%.

13. The hypodermic needle of claim 11, wherein the at least a portion of the external peripheral rim is beveled back at least 50%.

14. A method of preparing a sample, comprising withdrawing blood with the hypodermic needle of claim 1.

15. In a method of preparing a sample, comprising withdrawing blood with a hypodermic needle having an internal substantially cylindrical surface; an external substantially cylindrical surface; an end angled with respect to a longitudinal axis of the needle, the end having an opening and defining a piercing tip; an outer peripheral rim, the rim partially surrounding a first region of the opening proximal to the piercing tip and connecting the external and internal cylindrical surfaces of the needle; the improvement comprising withdrawing the blood with a hypodermic needle having an internal beveled surface on the internal surface of the needle partially surrounding a second region of said opening opposite the first region, wherein the degree of beveling back of the rim is at least 25%.

16. A method of making the hypodermic needle of claim 1, comprising:
beveling back an external peripheral rim of a hypodermic needle.

17. A method of making the hypodermic needle of claim 2, comprising:
beveling back an external peripheral rim of a hypodermic needle.

18. A method of making the hypodermic needle of claim 3, comprising:
beveling back an external peripheral rim of a hypodermic needle.

19. A method of making the hypodermic needle of claim 7, comprising:
beveling back an external peripheral rim of a hypodermic needle.

20. A method of making the hypodermic needle of claim 8, comprising:
beveling back an external peripheral rim of a hypodermic needle.
